(19) United States
(12) Patent Application Publication
(10) Pub. No.: US 2021/0177742 A1
(43) Pub. Date: Jun. 17, 2021

(54) LOADABLE POROUS STRUCTURES FOR USE AS IMPLANTS

(71) Applicant: Titan Pharmaceuticals, Inc., South San Francisco, CA (US)

(72) Inventor: Rajesh A. PATEL, Redwood City, CA (US)

(21) Appl. No.: 17/253,077

(22) PCT Filed: Jun. 25, 2019
(86) PCT No.: PCT/US2019/039070

§ 371 Date: Dec. 16, 2020

Related U.S. Application Data
(60) Provisional application No. 62/689,733, filed on Jun. 25, 2018.

(51) Int. Cl. A61K 9/00; A61K 47/32; A61K 47/38; A61K 47/14; A61K 47/12; A61K 47/02; B29C 48/00; B29C 67/20

(52) U.S. Cl. CPC A61K 9/0024; A61K 47/32; A61K 47/38; A61K 47/14; B29K 2023/083; A61K 47/02; B29C 48/022; B29C 67/202; A61K 47/12

(57) ABSTRACT

Loadable porous structures are disclosed, which are structures with pre-formed pores. The loadable porous structures can be loaded with pharmaceutical substances and optional excipients. The loaded porous structures can then be used as implants, for implantation into a patient for release of pharmaceutical substances over long periods of time. Methods of making and using such structures and implants are also disclosed.

### CROSS REFERENCE TO RELATED APPLICATIONS

This application claims priority benefit of U.S. Provisional Patent Appl. No. 62/689,733 filed Jun. 25, 2018. The entire contents of that application are hereby incorporated by reference herein.

### TECHNICAL FIELD

Provided are structures with pre-formed pores which can be loaded with pharmaceutical substances and implanted into a patient for release of pharmaceutical substances over long periods of time, as well as methods of making and using such structures.

### BACKGROUND OF THE INVENTION

Many patients require long-term, regular dosing with drugs or pharmaceutical substances. Several problems can arise during long-term administration of drugs taken orally or by other routes requiring frequent administration. Compliance with an extended dosing regimen can often be inconvenient or difficult. For example, patients with impaired cognitive function (due to Alzheimer's disease or other disorders) may not be able to self-administer drugs reliably, requiring a caregiver to ensure that medications are taken properly. Furthermore, enteral drug delivery is sometimes poorly tolerated or prohibited in patients with particular indications. Frequent or periodic administration, such as would occur with daily oral and sublingual delivery, can result in blood concentrations of drug peaking quickly after initial administration, then dropping steeply before the next administration. Intravenous drug delivery requires trained personnel for administration, and is impractical for prolonged outpatient treatment.

Implants used for drug delivery can overcome several problems with oral, sublingual, or intravenous administration of drugs. These implantable devices can produce long-term, continuous delivery of drugs, ensure compliance independent of the patient, maintain stable blood levels of medication, and reduce the likelihood of accidental use, abuse, or diversion for sale. Continuous release of a compound in vivo over an extended duration may be achieved via implantation of a device containing the compound encapsulated in a polymeric matrix. Examples of implantable polymeric devices for continuous drug release are described in, e.g., U.S. Pat. Nos. 4,883,666; 5,114,719; and 5,601,835. Patel et al. U.S. Pat. No. 7,736,665, U.S. Patent Application Publication Nos. 2004/0033250, 2007/0275031, and 2008/0026031, and Kleppner et al. 2006 J. Pharm. Pharmacol. 58:295-302 describe an implantable device comprising buprenorphine blended with ethylene vinyl acetate (EVA copolymer). Patel et al. U.S. Patent Application Publication No. 2005/0031668 describes an implantable polymeric device for sustained release of nalmefene. Patel et al. U.S. Patent Application Publication No. 2005/0031667 describes an implantable polymeric device for sustained release of dopamine agonists. Additional drug delivery devices include stents coated with compositions comprising drugs. Various devices and coatings are described in U.S. Pat. No. 6,506,437 to Harish; U.S. Pat. No. 7,364,748 to Claude; and U.S. Pat. No. 7,384,660 to Hossainy. U.S. Pat. No. 3,625,214 describes a drug-delivery device for prolonged drug delivery, fabricated in a spiral or “jellyroll” fashion. U.S. Pat. No. 3,926,188 describes a three-layer laminate drug dispenser comprising a core lamina of a crystalline drug of low water solubility dispersed in a polymer matrix, interposed between outer laminas made of a drug release rate controlling polymer. U.S. Pat. No. 5,683,719 describes a controlled release composition comprising an extruded core of active material and excipients, the core being coated in a water insoluble coating.

The current disclosure describes loadable implants suitable for use in a wide variety of applications.

### BRIEF SUMMARY OF THE INVENTION

Disclosed herein is a method of making a loadable porous structure, comprising extruding a mixture of a biocompatible matrix material and a porogen to form a matrix material-porogen extrudate; and removing the porogen from the extrudate to form the loadable porous structure. The matrix material can comprise a polymer, such as a non-biodegradable polymer. The matrix material can be selected from the group consisting of acrylics, agarose, alginate, cellulose ethers, collagen, copolymers containing poly(ethylene glycol) and polybutylene terephthalate segments (PEG/PBT) (PolyActive™), copolymers of poly(lactic) and glycolic acid, copolymers thereof with poly(ethylene glycol), derivatives and mixtures thereof, dextran, dextrose, elastin, epoxides, ethylene vinyl acetate (EVA copolymer), fluoropolymers, gelatin, hydroxypropylmethylcellulose, maleic anhydride copolymers, methyl cellulose and ethyl cellulose, non-water soluble cellulose acetate, non-water soluble chitosan, non-water soluble hydroxyethyl cellulose, non-water soluble hydroxypropyl cellulose, peptides, PLLA-poly-glycolic acid (PGA) copolymer (also known as poly-L-lactic acid-co-glycolic acid, or PLGA), poly (L-lactic acid), poly(2-ethoxyethyl methacrylate), poly(2-hydroxyethyl methacrylate), poly(2-methoxyethyl acrylate), poly(2-methoxyethyl methacrylate), poly(acrylamide), poly(alginic acid), poly(amino acids), poly(anhydrides), poly(aspartic acid), poly(benzyl glutamate), poly(beta-hydroxybutyrate), poly(caprolactone), poly(D,L-lactic acid), poly(D,L-lactide)(PLA), poly(D,L-lactide-co-caprolactone)(PLA/PCL) and poly(glycolide-co-caprolactone) (PGA/PCL), poly(D,L-lactide-co-glycolide) (PLA/PGA), poly(etherurethane urea), poly(ethyl glutamate-co-glutamic acid), poly(ethylene carbonate), poly(ethylene glycol), poly(ethylene-co-vinyl alcohol), poly(glutamic acid), poly(glutamic acid-co-ethyl glutamate), poly(glycolic acid), poly(glycolide-co-trimethylene carbonate) (PGA/PTMC), poly(hydroxypropyl methacrylamide), poly(imino carbonates), poly(leucine), poly(leucine-co-hydroxyethyl glutamine), poly(L-lactide-co-D,L-lactide) (PLLA/PLA), poly(L-lactide-co-glycolide)(PLLA/PGA), poly(lysine), poly(ortho esters), poly(orthoesters), poly(oxaamides), poly(oxaesters), poly(phosphate ester), poly(phosphazene), poly(phospho esters), poly(phosphoesters), poly(propylene carbonate), poly(propylene glycol), poly(pyrrole), poly(tert-butyloxy-carbonylmethyl glutamate), poly(tetramethylene glycol), poly(trimethylene carbonate), poly(ureas), poly(urethanes), poly(urethane-ureas), poly(vinyl alcohol), poly(vinyl alcohol-co-vinyl acetate), high molecular weight poly(vinylpyrrolidone) (PVP), poly[(97.5% dimethyl-trimethylene carbonate)-co-(2.5% trimethylene carbonate)], polyacrylic acid, polyalkylene oxides, polyamides, polycaprolactone (PCL) poly-(hydroxybutyrate-co-hydroxyvalerate) copolymer (PHBV), polycaprolactone (PCL), polycaprolactone co-butylacrylate, polydepsipeptides, polydioxanone (PDS), polyesters, polyethylene glycol, polyethylene oxide (PEO), polyethylene terephthalate (PET), polyglycolic acid and copolymers and mixtures thereof, poly(L-lactide) (PLLA), polyglycolic acid[polyglycolide (PGA)], polyhydroxybutyrate (PHBT) and copolymers of polyhydroxybutyrate, polyiminocarbonates, polylactic acid, polymethacrylic acid, polyolefins, polyphosphazene polymers, polypropylene fumarate, polysaccharides, hyaluronic acid, polytetrafluoroethylene (PTFE Teflon®), polyurethanes, silicones, tyrosine-derived polyarylates, tyrosine-derived polycarbonates, tyrosine-derived polyiminocarbonates, tyrosine-derived polyphosphonates, urethanes, polyamide, aliphatic polycarbonates, polyalkylcyanoacrylate, polyalkylene oxalates, polyanhydride, polycarboxylic acid, polyester, poly(hydroxybutyrate), polyimide, poly(iminocarbonate), polycaprolactone (PCL), poly-D,L-lactic acid (DL-PLA), polydioxanone, poly(glycolic acid), poly-L-lactic acid (L-PLA), poly-L-lactic acid-co-glycolic acid (PLGA), polyorthoester, polyphosphazenes, and polyphosphoester, poly(trimethylene carbonate), cellulose ester, polybutylene terephthalate, polycarbonate, polyester, polyether ether ketone, polyethylene-co-tetrafluoroethylene, polymethylmethacrylate, polyolefin, polypropylene, polysulfones, polytetrafluoroethylene, polyurethane, polyvinylchloride, polyvinylidene fluoride, silicone, ABS resins, acrylic polymers and copolymers, acrylonitrile-styrene copolymers, alkyd resins, carboxymethyl cellulose, ethylene-vinyl acetate copolymers, cellophane, cellulose butyrate, cellulose acetate butyrate, cellulose acetate, cellulose ethers, cellulose nitrate, cellulose propionate, copolymers of vinyl monomers with each other and olefins, ethylene-methyl methacrylate copolymers, epoxy resins, ethylene vinyl alcohol copolymer, poly(glyceryl sebacate), poly(glycolic acid-co-trimethylene carbonate), poly(hydroxybutyrate-co-valerate), poly(hydroxyvalerate), poly(lactide-co-glycolide), poly(propylene fumarate), poly(trimethylene carbonate), polyacrylonitrile, polyamides, Nylon 66, polycaprolactam, polycarbonates, polycyanoacrylates, polydioxanone, polyesters, polyethers, polyimides, polyisobutylene and ethylene-alphaolefin copolymers, polyoxymethylenes, polyphosphoester urethane, polyvinyl ketones, polyvinyl aromatics, polystyrene, polyvinyl esters, polyvinyl acetate, polyvinyl ethers, s polyvinyl methyl ether, polyvinylidene halides, vinylidene fluoride based homo- or co-polymer under the trade name Solef™ or Kynar™, polyvinylidene fluoride (PVDF), poly(vinylidene-co-hexafluoropropylene) (PVDF-co-HFP), polyvinylidene chloride, rayon, rayon-triacetate, silicones, vinyl halide polymers and copolymers, polyvinyl chloride, copolymers of these polymers with poly(ethylene glycol) (PEG), copolymers of poly(lactic) and glycolic acid, poly(anhydrides), poly(D,L-lactic acid), poly(D,L-lactide), poly(D,L-lactide-co-glycolide), poly(ethylene carbonate), poly(glycolic acid), poly(glycolide), poly(L-lactic acid), poly(L-lactide), poly(L-lactide-co-glycolide), poly(ortho esters), poly(oxaamides), poly(oxaesters), poly(phosphazenes), poly(phospho esters), poly(phosphoesters), poly(propylene carbonate), poly(trimethylene carbonate), poly(tyrosine derived carbonates), poly(tyrosine derived iminocarbonates), poly(tyrosine derived arylates), copolymers of these polymers with poly(ethylene glycol) (PEG), poly(ethylene-co-vinyl acetate) (EVA), polyvinylalcohol, polyurethanes, polycarbonate-based polyurethanes, and any combination or mixture of any two or more of the foregoing. The matrix material can be ethylene vinyl acetate (EVA).

The porogen can comprise a material selected from the group consisting of an alkyl cellulose, a hydroxyalkyl cellulose, ethylcellulose, methylcellulose, hydroxymethylcellulose, a fatty acid, stearic acid, palmitic acid, myristic acid, linoleic acid, a biocompatible salt, sodium chloride, calcium chloride, sodium phosphate, a solid organic acid, citric acid, a soluble polymer, and low molecular weight polyvinylpyrrolidone (low MW PVP). The porogen can comprise ethylcellulose or methylcellulose.

Removing the porogen from the extrudate can comprise treating the extrudate with a fluid that removes the porogen, such as by washing the extrudate with the fluid or immersing the extrudate in the fluid. The fluid can comprise water, saline, an aqueous buffer, an alcohol, ethanol, isopropanol, or supercritical carbon dioxide. In some embodiments, at least about 50% of the fluid-accessible porogen is removed from the extrudate. In some embodiments, the porogen is not a pharmaceutically active substance or drug.

The disclosure also provides loadable porous structure made by any of the methods disclosed herein, such as the methods described above.

The disclosure also provides a method of making a loaded porous structure, comprising forming a loadable porous structure by any method disclosed herein, such as the methods described above; loading a payload solution into pores of the loadable porous structure, where the payload solution comprises a solvent, a pharmaceutical substance, and optionally an excipient; and removing the solvent from the loadable porous structure to form the loaded porous structure. The steps of loading and removing can be repeated until the loaded porous structure contains a predetermined amount of pharmaceutical substance and optional excipient. The pharmaceutical substance can comprise a substance selected from the group consisting of a protein and a nucleic acid. The optional excipient can comprise a sugar alcohol, mannitol, glycerol, erythritol, threitol, arabitol, ribitol, xylitol, fucitol, galactitol, iditol, inositol, sorbitol, volemitol, isomalt, lactitol, maltitol, a biodegradable polymer, or poly (lactic-co-glycolic acid) (PLGA).

The disclosure also provides a loaded porous structure made by any of the methods disclosed herein, such as the methods described above.

The disclosure also provides a loadable porous structure, where the structure is prepared by a method comprising extruding a mixture of a biocompatible matrix material and a porogen to form a matrix material-porogen extrudate; and removing the porogen from the extrudate to form the loadable porous structure. A pharmaceutical substance can be loaded into the pores of the loadable porous structure to form a loaded porous structure. The pharmaceutical substance is optionally combined with an excipient prior to loading into the pores of the loadable porous structure.

The matrix material of the loadable porous structure or loaded porous structure can be a polymer. The matrix material of the loadable porous structure or loaded porous structure can be a non-biodegradable polymer. The matrix material can comprise a material selected from the group consisting of acrylics, agarose, alginate, cellulose ethers, collagen, copolymers containing poly(ethylene glycol) and polybutylene terephthalate segments (PEG/PBT) (PolyActive™), copolymers of poly(lactic) and glycolic acid, copolymers thereof with poly(ethylene glycol), derivatives and mixtures thereof, dextran, dextrose, elastin, epoxides, ethylene vinyl acetate (EVA copolymer), fluoropolymers, gelatin, hydroxypropylmethylcellulose, maleic anhydride copolymers, methyl cellulose and ethyl cellulose, non-water soluble cellulose acetate, non-water soluble chitosan, non-water soluble hydroxyethyl cellulose, non-water soluble hydroxypropyl cellulose, peptides, PLLA-poly-glycolic acid (PGA) copolymer (also known as poly-L-lactic acid-co-glycolic acid, or PLGA), poly (L-lactic acid), poly(2-ethoxyethyl methacrylate), poly(2-hydroxyethyl methacrylate), poly(2-methoxyethyl acrylate), poly(2-methoxyethyl methacrylate), poly(acrylamide), poly(alginic acid), poly(amino acids), poly(anhydrides), poly(aspartic acid), poly(benzyl glutamate), poly(beta-hydroxybutyrate), poly(caprolactone), poly(D,L-lactic acid), poly(D,L-lactide)(PLA), poly(D,L-lactide-co-caprolactone)(PLA/PCL) and poly(glycolide-co-caprolactone) (PGA/PCL), poly(D,L-lactide-co-glycolide) (PLA/PGA), poly(etherurethane urea), poly(ethyl glutamate-co-glutamic acid), poly(ethylene carbonate), poly(ethylene glycol), poly(ethylene-co-vinyl alcohol), poly(glutamic acid), poly(glutamic acid-co-ethyl glutamate), poly(glycolic acid), poly(glycolide-co-trimethylene carbonate) (PGA/PTMC), poly(hydroxypropyl methacrylamide), poly(imino carbonates), poly(leucine), poly(leucine-co-hydroxyethyl glutamine), poly(L-lactide-co-D,L-lactide) (PLLA/PLA), poly(L-lactide-co-glycolide)(PLLA/PGA), poly(lysine), poly(ortho esters), poly(orthoesters), poly(oxaamides), poly(oxaesters), poly(phosphate ester), poly(phosphazene), poly(phospho esters), poly(phosphoesters), poly(propylene carbonate), poly(propylene glycol), poly(pyrrole), poly(tert-butyloxy-carbonylmethyl glutamate), poly(tetramethylene glycol), poly(trimethylene carbonate), poly(ureas), poly(urethanes), poly(urethane-ureas), poly(vinyl alcohol), poly(vinyl alcohol-co-vinyl acetate), high molecular weight poly(vinylpyrrolidone) (PVP), poly[(97.5% dimethyl-trimethylene carbonate)-co-(2.5% trimethylene carbonate)], polyacrylic acid, polyalkylene oxides, polyamides, polycaprolactone (PCL) poly-(hydroxybutyrate-co-hydroxyvalerate) copolymer (PHBV), polycaprolactone (PCL), polycaprolactone co-butylacrylate, polydepsipeptides, polydioxanone (PDS), polyesters, polyethylene glycol, polyethylene oxide (PEO), polyethylene terephthalate (PET), polyglycolic acid and copolymers and mixtures thereof, poly(L-lactide) (PLLA), polyglycolic acid[polyglycolide (PGA)], polyhydroxybutyrate (PHBT) and copolymers of polyhydroxybutyrate, polyiminocarbonates, polylactic acid, polymethacrylic acid, polyolefins, polyphosphazene polymers, polypropylene fumarate, polysaccharides, hyaluronic acid, polytetrafluoroethylene (PTFE Teflon®), polyurethanes, silicones, tyrosine-derived polyarylates, tyrosine-derived polycarbonates, tyrosine-derived polyiminocarbonates, tyrosine-derived polyphosphonates, urethanes, polyamide, aliphatic polycarbonates, polyalkylcyanoacrylate, polyalkylene oxalates, polyanhydride, polycarboxylic acid, polyester, poly(hydroxybutyrate), polyimide, poly(iminocarbonate), polycaprolactone (PCL), poly-D,L-lactic acid (DL-PLA), polydioxanone, poly(glycolic acid), poly-L-lactic acid (L-PLA), poly-L-lactic acid-co-glycolic acid (PLGA), polyorthoester, polyphosphazenes, and polyphosphoester, poly(trimethylene carbonate), cellulose ester, polybutylene terephthalate, polycarbonate, polyester, polyether ether ketone, polyethylene-co-tetrafluoroethylene, polymethylmethacrylate, polyolefin, polypropylene, polysulfones, polytetrafluoroethylene, polyurethane, polyvinylchloride, polyvinylidene fluoride, silicone, ABS resins, acrylic polymers and copolymers, acrylonitrile-styrene copolymers, alkyd resins, carboxymethyl cellulose, ethylene-vinyl acetate copolymers, cellophane, cellulose butyrate, cellulose acetate butyrate, cellulose acetate, cellulose ethers, cellulose nitrate, cellulose propionate, copolymers of vinyl monomers with each other and olefins, ethylene-methyl methacrylate copolymers, epoxy resins, ethylene vinyl alcohol copolymer, poly(glyceryl sebacate), poly(glycolic acid-co-trimethylene carbonate), poly(hydroxybutyrate-co-valerate), poly(hydroxyvalerate), poly(lactide-co-glycolide), polypropylene fumarate), poly(trimethylene carbonate), polyacrylonitrile, polyamides, Nylon 66, polycaprolactam, polycarbonates, polycyanoacrylates, polydioxanone, polyesters, polyethers, polyimides, polyisobutylene and ethylene-alphaolefin copolymers, polyoxymethylenes, polyphosphoester urethane, polyvinyl ketones, polyvinyl aromatics, polystyrene, polyvinyl esters, polyvinyl acetate, polyvinyl ethers, s polyvinyl methyl ether, polyvinylidene halides, vinylidene fluoride based homo- or co-polymer under the trade name Solef™ or Kynar™, polyvinylidene fluoride (PVDF), poly(vinylidene-co-hexafluoropropylene) (PVDF-co-HFP), polyvinylidene chloride, rayon, rayon-triacetate, silicones, vinyl halide polymers and copolymers, polyvinyl chloride, copolymers of these polymers with poly(ethylene glycol) (PEG), copolymers of poly(lactic) and glycolic acid, poly(anhydrides), poly(D,L-lactic acid), poly(D,L-lactide), poly(D,L-lactide-co-glycolide), poly(ethylene carbonate), poly(glycolic acid), poly(glycolide), poly(L-lactic acid), poly(L-lactide), poly(L-lactide-co-glycolide), poly(ortho esters), poly(oxaamides), poly(oxaesters), poly(phosphazenes), poly(phospho esters), poly(phosphoesters), poly(propylene carbonate), poly(trimethylene carbonate), poly(tyrosine derived carbonates), poly(tyrosine derived iminocarbonates), poly(tyrosine derived arylates), copolymers of these polymers with poly(ethylene glycol) (PEG), poly(ethylene-co-vinyl acetate) (EVA), polyvinyl alcohol, polyurethanes, polycarbonate-based polyurethanes, and any combination or mixture of any two or more of the foregoing. The matrix material can be ethylene vinyl acetate (EVA).

The porogen used in the loadable porous structure can comprise a material selected from the group consisting of an alkyl cellulose, a hydroxyalkyl cellulose, ethylcellulose, methylcellulose, hydroxymethylcellulose, a fatty acid, stearic acid, palmitic acid, myristic acid, linoleic acid, a biocompatible salt, sodium chloride, calcium chloride, sodium phosphate, a solid organic acid, citric acid, a soluble polymer, and low molecular weight polyvinylpyrrolidone (low MW PVP). In embodiments, the porogen is not a pharmaceutically active substance or drug.

### BRIEF DESCRIPTION OF THE DRAWINGS

FIG. 1 shows a flow chart outlining the process of making the implants.

FIG. 2 shows a structure comprising a matrix containing porogenic substance.

FIG. 3 shows the structure of FIG. 2 after removal of most of the porogenic sub stance.

### DETAILED DESCRIPTION OF THE INVENTION

### Definitions and General Descriptions

“Drug” and “pharmaceutical substance” are equivalent terms and are used interchangeably, and encompasses any substance intended for therapeutic, diagnostic, or nutritional use in a patient, individual, or subject in need thereof. “Drugs” and “pharmaceutical substances” include, but are not limited to, diagnostic agents, therapeutic agents, hormones, nutrients, vitamins, and minerals.

“Porogen,” “porogenic material,” or “porogenic substance” are equivalent terms, and refer to a first material which is embedded or mixed into a second material, which can be removed (for example, by dissolution, diffusion, or degradation) from the second material. The removal of the porogen results in the creation of pores in the second material.

“Biocompatible,” when used to describe a material or system, indicates that the material or system does not provoke an adverse reaction, or causes only minimal, tolerable adverse reactions, when in contact with an organism, such as a human.

A “patient,” “individual,” or “subject” refers to a mammal, preferably a human, an agricultural animal such as a cow, pig, goat, or sheep, or a domestic animal such as a dog or cat. In a preferred embodiment, a patient, individual, or subject is a human.

“Treating” a disease or disorder with the implants and methods disclosed herein is defined as administering one or more of the implants disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the implants and methods disclosed herein is defined as administering one or more of the implants disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The implants and methods as disclosed herein can then be used in asymptomatic patients at risk of developing the clinical symptoms of the disease or disorder, in order to suppress the appearance of any adverse symptoms.

“Therapeutic use” of the implants disclosed herein is defined as using one or more of the implants disclosed herein to treat a disease or disorder, as defined above. A “therapeutically effective amount” of a drug, a pharmaceutical substance, or a therapeutic agent is an amount of the drug, pharmaceutical substance, or agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses. In the context of implantable devices, a therapeutically effective amount describes an amount released from the implant which is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. One or more implants can be used to deliver a therapeutically effective amount.

“Prophylactic use” of the implants disclosed herein is defined as using one or more of the implants disclosed herein to suppress a disease or disorder, as defined above. A “prophylactically effective amount” of a drug, pharmaceutical substance, or therapeutic agent is an amount of the drug, pharmaceutical substance, or agent, which, when administered to a patient, is sufficient to suppress the clinical manifestation of a disease or disorder, or to suppress the manifestation of adverse symptoms of a disease or disorder. A prophylactically effective amount can be administered to a patient as a single dose, or can be divided and administered as multiple doses. In the context of implantable devices, a prophylactically effective amount describes an amount released from the implant which is sufficient to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. One or more implants can be used to deliver a prophylactically effective amount.

“Blood level” as used herein refers to the concentration of a drug, pharmaceutical substance, therapeutic agent, hormone, metabolite, or other substance in the blood of a subject. A blood level can be measured in whole blood, blood serum, or blood plasma, as per standard clinical laboratory practice for the substance to be assayed.

As used herein, the singular forms “a”, “an”, and “the” include plural references unless indicated otherwise or the context clearly dictates otherwise.

A microporous material, as defined by the International Union of Pure and Applied Chemistry (IUPAC), has pores of size up to 2 nm. A mesoporous material has pores of size between 2 nm and 50 nm. A macroporous material has pores of size larger than 50 nm.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

With respect to numerical ranges disclosed in the present description, any disclosed upper limit for a component or parameter may be combined with any disclosed lower limit for that component or parameter to provide a range (provided that the upper limit is greater than the lower limit with which it is to be combined). Each of these combinations of disclosed upper and lower limits are explicitly envisaged herein. For example, if ranges for the amount of a particular component or parameter are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

The partition coefficient P of a compound is defined as the ratio of the concentration of the compound in organic solvent to the concentration of the compound in water, in a biphasic mixture of organic solvent and water (where the organic solvent and water are not miscible). The base-10 logarithm of the partition coefficient, log P, is often used. Partition coefficients are often measured in octanol/water systems, and the partition coefficient in such a system is defined as:

Poct=[concentration in octanol]÷[concentration in water].

For compounds which can ionize, the distribution coefficient D of a compound is defined as the ratio of the concentration of all species of the compound (ionized and unionized) in organic solvent to the concentration of all species of the compound (ionized and unionized) in water, in a biphasic mixture of organic solvent and water (where the organic solvent and water are not miscible). Log D may also be used. D will vary depending on the pH at which D is measured; preferably, D is measured at the physiological pH of 7.4. Distribution coefficients can be measured using octanol as the organic solvent. A solution of phosphate-buffered saline (PBS) at pH 7.4 can be used as the aqueous solvent when D is measured at physiological pH. (PBS comprises about 137 mM NaCl, about 2.7 mM KCl, about 10 mM Na₂HPO₄, and about 1.8 mM KH₂PO₄.)

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When an implant, device, composition, or system is described as “consisting essentially of” the listed elements, the implant, device, composition, or system contains the elements expressly listed, and may contain other elements which do not materially affect the condition being treated (for compositions for treating conditions), or the properties of the described implant, device, or system. However, the implant, device, composition, or system either does not contain any other elements which do materially affect the condition being treated other than those elements expressly listed (for compositions for treating systems) or does not contain any other elements which do materially affect the properties of the implant, device, or system; or, if the implant, device, composition, or system does contain extra elements other than those listed which may materially affect the condition being treated or the properties of the system, the implant, device, composition or system does not contain a sufficient concentration or amount of those extra elements to materially affect the condition being treated by the composition or the properties of the implant, device, or system. When a method is described as “consisting essentially of” the listed steps, the method contains the steps listed, and may contain other steps that do not materially affect the condition being treated by the method or the properties of the implant, device, or system produced by or used by the method, but the method does not contain any other steps which materially affect the condition being treated by the method or the implant, device, or system produced or used other than those steps expressly listed.

This disclosure provides several embodiments. It is contemplated that any features from any embodiment can be combined with any features from any other embodiment where possible. In this fashion, hybrid configurations of the disclosed features are within the scope of the present disclosure.

### General Principles of Loadable Porous Structures and Loaded Porous Structures for Use as Implants

Disclosed herein are implants for long-term sustained drug delivery. The implants comprise a matrix, a pharmaceutical substance or substances, and optionally one or more excipients. The implants are formed by combining the material used for the matrix with at least one type of porogen, and then removing the porogen, leaving behind pores in the matrix, to form a loadable porous structure. Hot melt extrusion can be used to combine the matrix material and the porogen. After removal of the porogen, the pores of the loadable porous structure can then be loaded with one or more pharmaceutical substances, and optionally one or more excipients, to prepare a loaded porous structure for use as the implant. When hot melt extrusion is used, loading of pharmaceutical substances and optional excipients will occur after the extrusion step, and thus the implants described herein are well-suited for use with pharmaceutical substances and excipients that are not stable at the elevated temperatures used for extrusion.

As will be appreciated, sufficient pores must be created in the matrix to form interconnected pores and channels. Simple extrusion of a polymer by itself, or simple solvent casting of a polymer by itself, will result in a solid polymeric structure without pores. However, inclusion of a material that can be removed from the extruded polymer or cast polymer can create a network of pores within the extrudate, to form a porous structure. As will also be appreciated, at least some of the pores in the matrix must open to the surface of the matrix in order for porogen to be extracted; for one or more pharmaceutical substances and, optionally, one or more excipients to be loaded into the pores; and for the one or more pharmaceutical substances and, optionally, one or more excipients to be released after the loaded porous structure is implanted into a patient.

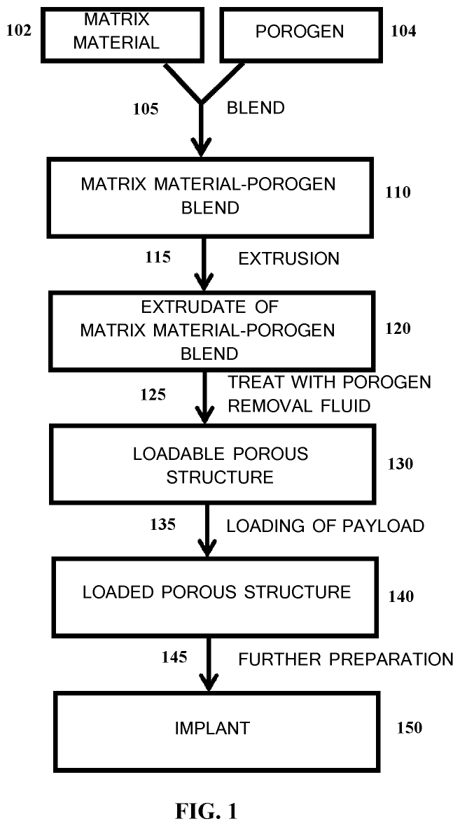

FIG. 1 shows a flow chart describing a method of making an implant as described herein. Matrix material 102 and porogen 104 are blended together at step 105 to form matrix material-porogen blend 110. The matrix material-porogen blend 110 is then extruded at step 115 via hot melt extrusion to form extrudate 120 of combined matrix material and porogen. The extrudate 120 is then treated with porogen removal fluid at step 125 to provide the loadable porous structure 130, which has porous matrix material from which the porogen has been removed. If sufficient porogen is not removed from the extrudate at step 125, step 125 can be repeated until sufficient porogen has been removed. At step 135, the payload, comprising one or more pharmaceutical substances, and optionally one or more excipients, is loaded into the loadable porous structure 130 to provide the loaded porous structure 140. If sufficient payload has not been loaded at step 135, step 135 can be repeated until sufficient payload is loaded. Further preparation, such as washing, drying, packaging, and sterilization, then takes place at step 145 to provide the implant 150.

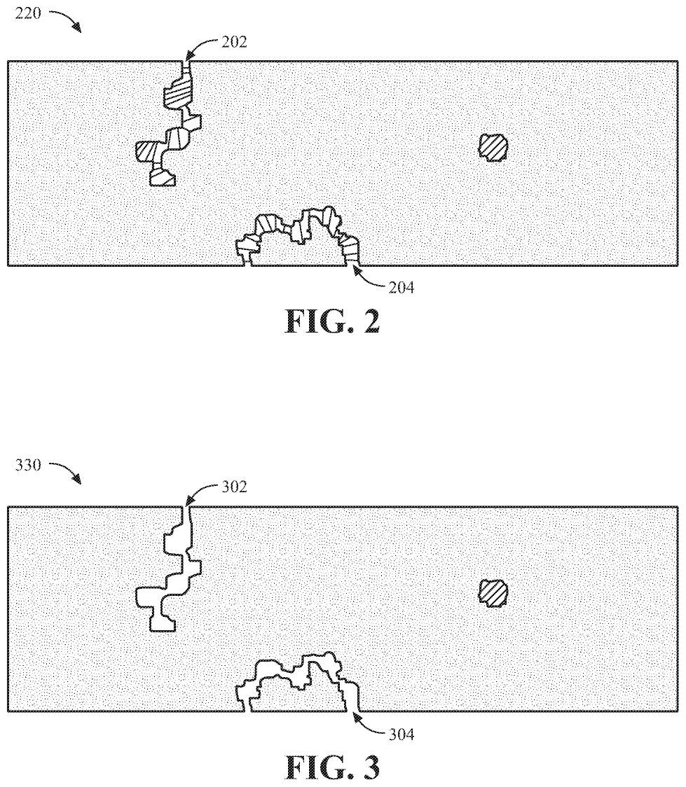

FIG. 2 shows an example of an extruded structure 220 (corresponding to 120 in FIG. 1) containing porogenic material. Regions 202 and 204 of the extruded structure comprise porogen (shown by hatched lines), while the black (unlabeled) portion of the structure comprises the matrix. Note that towards the right side of the figure, there is a small (unlabeled) region containing porogen which does not have a pathway to the surface of the structure 220.

Structure 330 in FIG. 3 (corresponding to 130 in FIG. 1) shows the extruded structure 220 of FIG. 2 after treatment to remove porogenic substance, such as by immersion in a fluid that removes the porogenic substance (for example, a solvent that dissolves the porogenic substance). Regions 302 and 304 are now empty pores resulting from the extraction of the porogen. Because the unlabeled region at the right of the figure did not have a pathway to connect to the surface of the structure, the fluid used to remove the porogen could not contact that region, and hence it remains filled with porogen (hatched lines). The porogenic substance in regions 202 and 204 of FIG. 2 could be accessed by the fluid for removal, and such material is referred to as fluid-accessible porogen. Material such as the porogenic substance in the unlabeled region at the right side of structure 220 of FIG. 2, which remains in the unlabeled region at the right side of structure 330 in FIG. 3, is referred to as fluid-inaccessible porogen.

By mixing appropriate amounts of matrix material and porogen, extruding them to form a matrix-porogen extrudate, and removing the porogen, a loadable porous structure can be formed with extensive porosity. The pores interconnect in a tortuous manner within the bulk of the structure; that is, the pores interconnect by repeatedly bending, twisting, and changing directions. Only a small fraction of the pores that are present in the loadable porous structure are depicted in FIG. 3; in practice, there will be an extensive network of interconnecting channels and pores.

The loadable porous structure 130 of FIG. 1, such as loadable porous structure 330 of FIG. 3, can then be loaded with one or more pharmaceutical substances, and optionally one or more excipients, as described herein, to result in a loaded porous structure. This loaded porous structure can then be used as an implant in a subject, patient, or individual, in order to deliver the one or more pharmaceutical substances to the subject, patient, or individual.

### Physical Parameters of Porous Structures and Implants

In some embodiments, the loadable porous structures, loaded porous structures, and implants disclosed herein are rod-shaped or generally rod-shaped, and are about 0.5 cm to 10 cm in length, such as from about 1 cm to about 6 cm in length, or from about 1 cm to about 5 cm in length, or about 1 cm to about 4 cm in length, or about 1 cm to 3 cm in length, or about 1.5 cm to 3.5 cm in length, or about 2 cm to 4 cm in length, or about 2 cm to about 3 cm in length, or about 2 cm to about 5 cm in length, or about 2 cm to about 6 cm in length, or about 3 cm to about 5 cm in length, or about 3 cm to about 6 cm in length, or about 4 cm to about 5 cm in length, or about 4 cm to about 6 cm in length, or about 2.6 cm in length. In some embodiments, the loadable porous structures, loaded porous structures, and implants are rod-shaped or generally rod-shaped, and are about 3 cm to about 5 cm in length, or about 3.5 cm to about 4.5 cm, or about 4 cm. In some embodiments, the loadable porous structures, loaded porous structures, and implants are rod-shaped or generally rod-shaped, and are about 5 cm to about 7 cm in length, or about 5.5 cm to about 6.5 cm, or about 6 cm.

In some embodiments, the loadable porous structures, loaded porous structures, and implants are rod-shaped or generally rod-shaped, and are about 1 to about 3 mm in diameter. In some embodiments, the loadable porous structures, loaded porous structures, and implants are rod-shaped or generally rod-shaped, and comprise dimensions of about 0.5 to about 7 mm in diameter, or about 2 to about 5 mm in diameter, or about 2 to about 3 mm in diameter, or about 2.4 mm in diameter, or about 3 mm in diameter.

Any of the recited lengths can be combined with any of the recited diameters. In some embodiments, the loadable porous structures, loaded porous structures, and implants are rod-shaped or generally rod-shaped, and comprise dimensions of about 2.4 mm in total diameter and about 2.6 cm in total length.

### Chemical Composition of Loadable Porous Structures, Loaded Porous Structures, and Implants

The loadable porous structures, loaded porous structures, and implants described herein can be formulated from any biocompatible substance that can be implanted into a subject, patient, or individual. The portion of the loaded porous structure or implant which serves as a carrier for the pharmaceutical substance, the excipient(s), and any other substances included in the loaded porous structure or implant, is referred to as the matrix or the matrix material.

Polymers can be used as the matrix material. One such matrix is the polymer ethylene vinyl acetate (EVA). EVA is a co-polymer of the monomers ethylene and vinyl acetate. The composition of EVA is usually specified as the percent by weight of vinyl acetate present, with the remaining percentage made up of ethylene. Various ratios of the monomers can be used, such as about 10% to about 50% vinyl acetate by weight, with the remainder being ethylene; about 20% to about 45% vinyl acetate; about 25% to about 40% vinyl acetate; about 30% to about 36% vinyl acetate, or about 33% vinyl acetate.

In some embodiments as disclosed herein, the implants additionally comprise a radiopaque substance. The radiopaque substance is preferably opaque to X-ray radiation. The radiopaque substance aids in precisely locating the implant in a non-invasive manner, for example, in an X-ray or CT scan. Barium salts, such as barium sulfate, are preferred radiopaque substances. Other radiopaque substances which can be used include, but are not limited to, zirconium oxide, bismuth oxide, bismuth salts, and tungsten compounds such as calcium tungstate.

In some embodiments as disclosed herein, the implants additionally comprise a substance which is detectable or identifiable by magnetic resonance imaging, for use in locating the implant during an MRI scan. Iron oxides, such as paramagnetic iron oxide (Fe₃O₄), can be used as a substance to visualize implants in an MRI scan.

In some embodiments as disclosed herein, the implants additionally comprise both a radiopaque substance and a substance which is detectable by magnetic resonance imaging.

The detectable substance or substances can be blended into the matrix of the implant if such blending does not substantially affect the preparation of the implant or the pharmacokinetics of drug release. Alternatively, the detectable substance can be restricted to a particular location of the implant where it will not interfere with the preparation of the implant or the pharmacokinetics of drug release, such as in the core of the implant, or at one or both end regions of the implant.

### Pharmaceutical Substance and Drugs for Use in Implants

A variety of pharmaceutical substances and drugs can be loaded into the loadable porous structures to prepare the loaded porous structures and implants disclosed herein. Since the pharmaceutical substance or drug will not be exposed to the elevated temperatures used during extrusion processes, the implants are particularly useful for delivery of temperature-sensitive drugs. Temperature-sensitive drugs include, but are not limited to, proteins and nucleic acids. Drugs which must be kept refrigerated (so-called “cold chain drugs” and “cool-chain drugs”) can be loaded into the loadable porous structures, under appropriate temperatures and other conditions which maintain the stability of the drug. Drugs contained in loaded porous structures for use as pharmaceutical substance-containing implants should also be sufficiently stable over the period that the implant remains in the patient. For human patients, the implant will be in an environment at or near body temperature of 37° C.

One group of temperature-sensitive drugs comprises antibodies, engineered antibody variants, and antibody fragments. In some embodiments, a monoclonal antibody can be loaded into the loadable porous structures. In some embodiments, antigen-binding fragments (Fab), single chain variable fragments (scFv), Fc regions, antigen-binding Fc regions (Fcab), single domain antibodies (sdAb), bispecific antibodies (bsAb or BiAb), and multispecific antibodies (msAb, such as the trispecific TriMab) can be loaded into the loadable porous structures.

Examples of drugs which should be kept refrigerated are described on the following list, where the trademarked brand name is followed by the generic name in parentheses and a short description: ORENCIA® (abatacept), fusion protein of the extracellular domain of human cytotoxic T-lymphocyte-associated antigen 4 linked to the modified Fc (hinge, CH2, and CH3 domains) portion of human immunoglobulin G1; HUMIRA® (adalimumab), recombinant human IgG1 monoclonal antibody; KINERET® (anakinra), recombinant, nonglycosylated form of the human interleukin-1 receptor antagonist (IL-1Ra); ENBREL® (etanercept), dimeric fusion protein consisting of the extracellular ligand-binding portion of the human 75 kilodalton (p′75) tumor necrosis factor receptor (TNFR) linked to the Fc portion of human IgG1; INFLECTRA® (infliximab-dyyb), chimeric IgG1κ monoclonal antibody (composed of human constant and murine variable regions) specific for human tumor necrosis factor-alpha (TNFα); REMSIMA® (infliximab), chimeric human-murine IgG1 monoclonal antibody produced in murine hybridoma cells by recombinant DNA technology; SIMPONI® (gollmumab), a human IgG 1κ monoclonal antibody specific for human tumor necrosis factor alpha (TNFα) that exhibits multiple glycoforms; REMICAD®E (infliximab), chimeric IgG1κ monoclonal antibody (composed of human constant and murine variable regions) specific for human tumor necrosis factor-alpha (TNFα); ENTYVIO® (vedolizumab), integrin receptor antagonist, is a humanized IgG1 monoclonal antibody produced in Chinese hamster ovary cells that binds to the human α4β7 integrin; STELARA® (ustekinumab), human IgG1κ monoclonal antibody against the p40 subunit of the IL-12 and IL-23 cytokines LEMTRADA® (alemtuzumab), recombinant humanized IgG1 kappa monoclonal antibody directed against the cell surface glycoprotein, CD52; TYSABRI® (natalizumab), recombinant humanized IgG4κ monoclonal antibody produced in murine myeloma cells. Natalizumab contains human framework regions and the complementarity-determining regions of a murine antibody that binds to α4-integrin; HUMALOG® (insulin lispro injection), rapid-acting human insulin analog used to lower blood glucose; differs from human insulin in that the amino acid proline at position B28 is replaced by lysine and the lysine in position B29 is replaced by proline. Chemically, it is Lys(B28), Pro(B29) human insulin analog; INSUMAN BASAL® (insulin for injection), insulin suspensions; LANTUS® (insulin glargine injection), recombinant human insulin analog that is a long-acting, parenteral blood-glucose-lowering agent; differs from human insulin in that the amino acid asparagine at position A21 is replaced by glycine and two arginines are added to the C-terminus of the B-chain; OMNITROPE® (somatropin), polypeptide hormone of recombinant DNA origin; amino acid sequence of the product is identical to that of human growth hormone of pituitary origin; GENOTROPIN® (somatropin), lyophilized powder [that] contains somatropin, which is a polypeptide hormone of recombinant DNA origin; amino acid sequence of the product is identical to that of human growth hormone of pituitary origin (somatropin); and HUMATROPE® (somatropin), polypeptide hormone of recombinant DNA origin.

Implants can contain a single drug or pharmaceutical substance, and optionally, an excipient or excipients. Implants can contain two drugs or two pharmaceutical substances, and optionally, an excipient or excipients. Implants can contain multiple drugs or multiple pharmaceutical substances, and optionally, an excipient or excipients.

### Excipients for Use in Implants

Excipients can optionally be used in the implants along with pharmaceutical substances. Mixtures of any two or more of the excipients recited herein can also be used.

Sugar alcohols that can be used as excipients in the implants include, but are not limited to, mannitol, glycerol, erythritol, threitol, arabitol, ribitol, xylitol, fucitol, galactitol, iditol, inositol, sorbitol, volemitol, isomalt, lactitol, and maltitol. A subset of sugar alcohols that can be used comprises the six-carbon compounds mannitol, fucitol, galactitol, iditol, inositol, and sorbitol. In one embodiment, mannitol is used as the excipient.

Polymers that can be used as excipients in the implants include, but are not limited to, poly (lactic-co-glycolic acid) (PLGA), erodible or bioerodible forms of polyamide, aliphatic polycarbonates, polyalkylcyanoacrylates, polyalkylene oxalates, polyanhydrides, polycarboxylic acids, polyesters, poly(hydroxybutyrate), polyimides, poly(iminocarbonates), polycaprolactone (PCL), poly-D,L-lactic acid (DL-PLA), polydioxanone, poly(glycolic acid), poly-L-lactic acid (L-PLA), polyorthoesters, polyphosphazenes, polyphosphoesters, poly(trimethylene carbonate), and derivatives and mixtures thereof.

Additional polymers that can be used as excipients in the implants include, but are not limited to, cellulose ester, polybutylene terephthalate, polycarbonate, polyester, polyether ether ketone, polyethylene-co-tetrafluoroethylene, polymethylmethacrylate, polyolefin, polypropylene, polysulfones, polytetrafluoroethylene, polyurethane, polyvinylchloride, polyvinylidene fluoride, silicone, and derivatives and combinations thereof.

The excipient:drug ratio can range from 5:1 to 1:10 by weight, such as 1:1 to 1:6 (for example, 1:1, 1:2, 1:3, 1:4, 1:5, or 1:6), or 1:3 to 1:6. In one embodiment, the excipient:drug ratio is 1:4.

### Exemplary Polymers for Use in Implants

A preferred polymer for use in the implants is ethylene vinyl acetate (EVA; poly(ethylene-co-vinyl acetate)). However, other biocompatible polymers can be used in the implants disclosed herein. As used herein, a “polymer” or “polymeric material” means a macromolecule comprising repeating monomer units or co-monomer units. The polymer may be bioerodible or non-bioerodible. The polymer may be a homopolymer, copolymer, terpolymer, or may contain more than three monomers. The polymer is preferably biocompatible.

Exemplary polymers that can be used for making implants include, but are not limited to: acrylics, agarose, alginate, and combinations, cellulose ethers, collagen, copolymers containing poly(ethylene glycol) and polybutylene terephthalate segments (PEG/PBT) (PolyActive™), copolymers of poly(lactic) and glycolic acid, copolymers thereof with poly(ethylene glycol), derivatives and mixtures thereof, dextran, dextrose, elastin, epoxides, ethylene vinyl acetate (EVA copolymer), fluoropolymers, gelatin, hydroxypropylmethylcellulose, maleic anhydride copolymers, methyl cellulose and ethyl cellulose, non-water soluble cellulose acetate, non-water soluble chitosan, non-water soluble hydroxyethyl cellulose, non-water soluble hydroxypropyl cellulose, peptides, PLLA-poly-glycolic acid (PGA) copolymer (also known as poly-L-lactic acid-co-glycolic acid, or PLGA), poly (L-lactic acid), poly(2-ethoxyethyl methacrylate), poly(2-hydroxyethyl methacrylate), poly(2-methoxyethyl acrylate), poly(2-methoxyethyl methacrylate), poly(acrylamide), poly(alginic acid), poly(amino acids), poly(anhydrides), poly(aspartic acid), poly(benzyl glutamate), poly(beta-hydroxybutyrate), poly(caprolactone), poly(D,L-lactic acid), poly(D,L-lactide)(PLA), poly(D,L-lactide-co-caprolactone)(PLA/PCL) and poly(glycolide-co-caprolactone) (PGA/PCL), poly(D,L-lactide-co-glycolide) (PLA/PGA), poly(etherurethane urea), poly(ethyl glutamate-co-glutamic acid), poly(ethylene carbonate), poly(ethylene glycol), poly(ethylene-co-vinyl alcohol), poly(glutamic acid), poly(glutamic acid-co-ethyl glutamate), poly(glycolic acid), poly(glycolide-co-trimethylene carbonate) (PGA/PTMC), poly(hydroxypropyl methacrylamide), poly(imino carbonates), poly(leucine), poly(leucine-co-hydroxyethyl glutamine), poly(L-lactide-co-D,L-lactide) (PLLA/PLA), poly(L-lactide-co-glycolide)(PLLA/PGA), poly(lysine), poly(ortho esters), poly(orthoesters), poly(oxaamides), poly(oxaesters), poly(phosphate ester), poly(phosphazene), poly(phospho esters), poly(phosphoesters), poly(propylene carbonate), poly(propylene glycol), poly(pyrrole), poly(tert-butyloxy-carbonylmethyl glutamate), poly(tetramethylene glycol), poly(trimethylene carbonate), poly(ureas), poly(urethanes), poly(urethane-ureas), poly(vinyl alcohol), poly(vinyl alcohol-co-vinyl acetate), high molecular weight poly(vinylpyrrolidone) (PVP), poly[(97.5% dimethyl-trimethylene carbonate)-co-(2.5% trimethylene carbonate)], polyacrylic acid, polyalkylene oxides, polyamides, polycaprolactone (PCL) poly-(hydroxybutyrate-co-hydroxyvalerate) copolymer (PHBV), polycaprolactone (PCL), polycaprolactone co-butylacrylate, polydepsipeptides, polydioxanone (PDS), polyesters, polyethylene glycol, polyethylene oxide (PEO), polyethylene terephthalate (PET), polyglycolic acid and copolymers and mixtures thereof such as poly(L-lactide) (PLLA), polyglycolic acid[polyglycolide (PGA)], polyhydroxybutyrate (PHBT) and copolymers of polyhydroxybutyrate, polyiminocarbonates, polylactic acid, polymethacrylic acid, polyolefins, polyphosphazene polymers, polypropylene fumarate, polysaccharides such as hyaluronic acid, polytetrafluoroethylene (PTFE Teflon®), polyurethanes, silicones, tyrosine-derived polyarylates, tyrosine-derived polycarbonates, tyrosine-derived polyiminocarbonates, tyrosine-derived polyphosphonates, urethanes, and combinations, derivatives and mixtures thereof.

Exemplary erodible or bioerodible polymers that can be used for making implants include, but are not limited to, erodible or bioerodible forms of polyamide, aliphatic polycarbonates, polyalkylcyanoacrylate, polyalkylene oxalates, polyanhydride, polycarboxylic acid, polyester, poly(hydroxybutyrate), polyimide, poly(iminocarbonate), polycaprolactone (PCL), poly-D,L-lactic acid (DL-PLA), polydioxanone, poly(glycolic acid), poly-L-lactic acid (L-PLA), poly-L-lactic acid-co-glycolic acid (PLGA), polyorthoester, polyphosphazenes, and polyphosphoester, poly(trimethylene carbonate), and derivatives and mixtures thereof.

The implants may also be formed from a material selected from the group consisting of cellulose ester, polybutylene terephthalate, polycarbonate, polyester, polyether ether ketone, polyethylene-co-tetrafluoroethylene, polymethylmethacrylate, polyolefin, polypropylene, polysulfones, polytetrafluoroethylene, polyurethane, polyvinylchloride, polyvinylidene fluoride, silicone, and derivatives and combinations thereof.

Additional representative examples of the polymer for use in the implants disclosed herein include, but are not limited to, ABS resins, acrylic polymers and copolymers, acrylonitrile-styrene copolymers, alkyd resins, and carboxymethyl cellulose, and ethylene-vinyl acetate copolymers, cellophane, cellulose butyrate, cellulose acetate butyrate, cellulose acetate, cellulose ethers, cellulose nitrate, cellulose propionate, copolymers of vinyl monomers with each other and olefins, ethylene-methyl methacrylate copolymers, epoxy resins, ethylene vinyl alcohol copolymer (commonly known by the generic name EVOH or by the trade name EVAL), poly(glyceryl sebacate), poly(glycolic acid-co-trimethylene carbonate), poly(hydroxybutyrate-co-valerate), poly(hydroxyvalerate), poly(lactide-co-glycolide), poly(propylene fumarate), poly(trimethylene carbonate), polyacrylonitrile, polyamides, such as Nylon 66 and polycaprolactam, polycarbonates, polycyanoacrylates, polydioxanone, polyesters, polyethers, polyimides, polyisobutylene and ethylene-alphaolefin copolymers, polyoxymethylenes, polyphosphoester urethane, polyvinyl ketones, polyvinyl aromatics, such as polystyrene, polyvinyl esters, such as polyvinyl acetate, polyvinyl ethers, such as polyvinyl methyl ether, polyvinylidene halides, such as vinylidene fluoride based homo- or co-polymer under the trade name Solef™ or Kynar™, for example, polyvinylidene fluoride (PVDF) or poly(vinylidene-co-hexafluoropropylene) (PVDF-co-HFP) and polyvinylidene chloride, rayon, rayon-triacetate, silicones, vinyl halide polymers and copolymers, such as polyvinyl chloride, copolymers of these polymers with poly(ethylene glycol) (PEG), or combinations thereof

In some embodiments, the polymer can be copolymers of poly(lactic) and glycolic acid, poly(anhydrides), poly(D,L-lactic acid), poly(D,L-lactide), poly(D,L-lactide-co-glycolide), poly(ethylene carbonate), poly(glycolic acid), poly(glycolide), poly(L-lactic acid), poly(L-lactide), poly(L-lactide-co-glycolide), poly(ortho esters), poly(oxaamides), poly(oxaesters), poly(phosphazenes), poly(phospho esters), poly(phosphoesters), polypropylene carbonate), poly(trimethylene carbonate), poly(tyrosine derived carbonates), poly(tyrosine derived iminocarbonates), poly(tyrosine derived arylates), copolymers of these polymers with poly(ethylene glycol) (PEG), or combinations thereof.

Examples of non-bioerodible polymers useful in the implants disclosed herein include, but are not limited to, poly(ethylene-co-vinyl acetate) (EVA), polyvinylalcohol and polyurethanes, such as polycarbonate -based polyurethanes.

As previously noted, a preferred polymer for the implants is ethylene vinyl acetate (EVA).

The implants can comprise a single type of polymer or a mixture of two or more polymers. A mixture of two polymers may modulate the release rate of the drug. It is desirable that an effective therapeutic amount of the drug be released from any implant as disclosed herein for a reasonably long period of time. U.S. Pat. No. 6,258,121 to Yang et al. disclosed a method of altering the release rate by blending two polymers with differing release rates and incorporating them into a single layer; this technique can also aid in reducing burst release of drug upon implant.

### Exemplary Porogens

Examples of porogens which can be used include, but are not limited to, alkyl celluloses and hydroxyalkyl celluloses, such as ethylcellulose, methylcellulose, and hydroxymethylcellulose; fatty acids such as stearic acid, palmitic acid, myristic acid, and linoleic acid; biocompatible salts, such as sodium chloride, calcium chloride, or sodium phosphate; solid organic acids such as citric acid; and soluble polymers such as low molecular weight polyvinylpyrrolidone (PVP). Porogen particles are preferably used in a tight size distribution to enable control over the size of the pores. The mean diameter of the porogens used can be between about 1 micrometer and about 300 micrometers. In some embodiments, the mean diameter of the porogens is about 5% of the diameter of the implant.

The porogens (also referred to as porogenic materials or porogenic substances) function to create pores in the matrix material of the loadable porous structures, loaded porous structures, and implants, and in preferred embodiments, the porogens are not pharmaceutically active substances or drugs. In alternate preferred embodiments, porogens are not pharmaceutically active substances or drugs for the disease or condition which the implant is intended to treat. Thus, for example, when the porogen is citric acid, the implant is not intended to treat a disease or condition for which citric acid is useful for treatment.

In some embodiments, the porogen material comprises spherical particles or approximately spherical particles, and at least about 90% of the particles have a diameter between about 1 micrometer and about 50 micrometers. In some embodiments, the porogen material comprises spherical particles or approximately spherical particles, with a mean diameter between about 1 micrometer and about 50 micrometers. In some embodiments, the porogen material comprises spherical particles or approximately spherical particles, and at least about 90% of the particles have a diameter that varies by about 10% or less from a mean diameter.

In some embodiments, the porogen material comprises particles and the longest dimension of at least about 90% of the particles is between about 1 micrometer and about 50 micrometers. In some embodiments, the porogen material comprises particles and the longest dimension of the particles is between about 1 micrometer and about 50 micrometers. In some embodiments, the porogen material comprises particles and the longest dimension of at least about 90% of the particles varies by 10% or less from the average longest dimension of the particles.

In some embodiments, the porogen material comprises particles and the mean dimension of at least about 90% of the particles is between about 1 micrometer and about 50 micrometers, where the mean dimension of the particles is the mean of the longest dimension of the particles and the shortest dimension of the particles. In some embodiments, the porogen material comprises particles and the mean dimension of the particles is between about 1 micrometer and about 50 micrometers. In some embodiments, the porogen material comprises particles and the mean dimension of at least about 90% of the particles varies by 10% or less from the average of the mean dimension of the particles.

The mean diameter of the porogen particles, such as spherical particles or approximately spherical particles, can be between about 1 micrometer and about 300 micrometers. In one embodiment, at least about 90% of the particles have a diameter that varies by about 10% or less from a mean diameter, where the mean diameter is between about 1 micrometer and about 300 micrometers. In one embodiment, at least about 90% of the particles have a diameter that varies by about 10% or less from a mean diameter, where the mean diameter is between about 1 micrometer and about 200 micrometers. In one embodiment, at least about 90% of the particles have a diameter that varies by about 10% or less from a mean diameter, where the mean diameter is between about 1 micrometer and about 100 micrometers. In one embodiment, at least about 90% of the particles have a diameter that varies by about 10% or less from a mean diameter, where the mean diameter is between about 1 micrometer and about 50 micrometers. In one embodiment, at least about 90% of the particles have a diameter that varies by about 10% or less from a mean diameter, where the mean diameter is between about 1 micrometer and about 30 micrometers. In one embodiment, at least about 90% of the particles have a diameter that varies by about 10% or less from a mean diameter, where the mean diameter is between about 1 micrometer and about 25 micrometers. In one embodiment, at least about 90% of the particles have a diameter that varies by about 10% or less from a mean diameter, where the mean diameter is between about 1 micrometer and about 20 micrometers. In one embodiment, at least about 90% of the particles have a diameter that varies by about 10% or less from a mean diameter, where the mean diameter is between about 1 micrometer and about 10 micrometers. In one embodiment, at least about 90% of the particles have a diameter that varies by about 10% or less from a mean diameter, where the mean diameter is between about 1 micrometer and about 5 micrometers.

In one embodiment, at least about 75% of the particles have a diameter less than about 300 micrometers. In one embodiment, at least about 75% of the particles have a diameter less than about 200 micrometers. In one embodiment, at least about 75% of the particles have a diameter less than about 100 micrometers. In one embodiment, at least about 75% of the particles have a diameter less than about 50 micrometers. In one embodiment, at least about 75% of the particles have a diameter less than about 30 micrometers. In one embodiment, at least about 75% of the particles have a diameter less than about 25 micrometers. In one embodiment, at least about 75% of the particles have a diameter less than about 20 micrometers. In one embodiment, at least about 75% of the particles have a diameter less than about 10 micrometers. In one embodiment, at least about 75% of the particles have a diameter less than about 5 micrometers.

In one embodiment, at least about 90% of the particles have a diameter less than about 300 micrometers. In one embodiment, at least about 90% of the particles have a diameter less than about 200 micrometers. In one embodiment, at least about 90% of the particles have a diameter less than about 100 micrometers. In one embodiment, at least about 90% of the particles have a diameter less than about 50 micrometers. In one embodiment, at least about 90% of the particles have a diameter less than about 30 micrometers. In one embodiment, at least about 90% of the particles have a diameter less than about 25 micrometers. In one embodiment, at least about 90% of the particles have a diameter less than about 20 micrometers. In one embodiment, at least about 90% of the particles have a diameter less than about 10 micrometers. In one embodiment, at least about 90% of the particles have a diameter less than about 5 micrometers.

For particles which are non-spherical or irregularly shaped, such as needle-type particles, the particles can be characterized by their longest dimension. The mean longest dimension of the porogens can be between about 1 micrometer and about 300 micrometers. In one embodiment, at least about 90% of the particles have a longest dimension that varies by about 10% or less from a mean longest dimension, where the mean longest dimension is between about 1 micrometer and about 300 micrometers. In one embodiment, at least about 90% of the particles have a longest dimension that varies by about 10% or less from a mean longest dimension, where the mean longest dimension is between about 1 micrometer and about 200 micrometers. In one embodiment, at least about 90% of the particles have a longest dimension that varies by about 10% or less from a mean longest dimension, where the mean longest dimension is between about 1 micrometer and about 100 micrometers. In one embodiment, at least about 90% of the particles have a longest dimension that varies by about 10% or less from a mean longest dimension, where the mean longest dimension is between about 1 micrometer and about 50 micrometers. In one embodiment, at least about 90% of the particles have a longest dimension that varies by about 10% or less from a mean longest dimension, where the mean longest dimension is between about 1 micrometer and about 30 micrometers. In one embodiment, at least about 90% of the particles have a longest dimension that varies by about 10% or less from a mean longest dimension, where the mean longest dimension is between about 1 micrometer and about 25 micrometers. In one embodiment, at least about 90% of the particles have a longest dimension that varies by about 10% or less from a mean longest dimension, where the mean longest dimension is between about 1 micrometer and about 20 micrometers. In one embodiment, at least about 90% of the particles have a longest dimension that varies by about 10% or less from a mean longest dimension, where the mean longest dimension is between about 1 micrometer and about 10 micrometers. In one embodiment, at least about 90% of the particles have a longest dimension that varies by about 10% or less from a mean longest dimension, where the mean longest dimension is between about 1 micrometer and about 5 micrometers.

In one embodiment, at least about 75% of the particles have a longest dimension less than about 300 micrometers. In one embodiment, at least about 75% of the particles have a longest dimension less than about 200 micrometers. In one embodiment, at least about 75% of the particles have a longest dimension less than about 100 micrometers. In one embodiment, at least about 75% of the particles have a longest dimension less than about 50 micrometers. In one embodiment, at least about 75% of the particles have a longest dimension less than about 30 micrometers. In one embodiment, at least about 75% of the particles have a longest dimension less than about 25 micrometers. In one embodiment, at least about 75% of the particles have a longest dimension less than about 20 micrometers. In one embodiment, at least about 75% of the particles have a longest dimension less than about 10 micrometers. In one embodiment, at least about 75% of the particles have a longest dimension less than about 5 micrometers.

In one embodiment, at least about 90% of the particles have a longest dimension less than about 300 micrometers. In one embodiment, at least about 90% of the particles have a longest dimension less than about 200 micrometers. In one embodiment, at least about 90% of the particles have a longest dimension less than about 100 micrometers. In one embodiment, at least about 90% of the particles have a longest dimension less than about 50 micrometers. In one embodiment, at least about 90% of the particles have a longest dimension less than about 30 micrometers. In one embodiment, at least about 90% of the particles have a longest dimension less than about 25 micrometers. In one embodiment, at least about 90% of the particles have a longest dimension less than about 20 micrometers. In one embodiment, at least about 90% of the particles have a longest dimension less than about 10 micrometers. In one embodiment, at least about 90% of the particles have a longest dimension less than about 5 micrometers.

For particles which are non-spherical or irregularly shaped, such as needle-type particles, the particles can be also characterized by the mean of their longest dimension and shortest dimension (“mean of LD and SD”). The average mean of LD and SD of the porogens can be between about 1 micrometer and about 300 micrometers. In one embodiment, at least about 90% of the particles have a mean of LD and SD that varies by about 10% or less from an average mean of LD and SD, where the average mean of LD and SD is between about 1 micrometer and about 300 micrometers. In one embodiment, at least about 90% of the particles have a mean of LD and SD that varies by about 10% or less from an average mean of LD and SD, where the average mean of LD and SD is between about 1 micrometer and about 200 micrometers. In one embodiment, at least about 90% of the particles have a mean of LD and SD that varies by about 10% or less from an average mean of LD and SD, where the average mean of LD and SD is between about 1 micrometer and about 100 micrometers. In one embodiment, at least about 90% of the particles have a mean of LD and SD that varies by about 10% or less from an average mean of LD and SD, where the average mean of LD and SD is between about 1 micrometer and about 50 micrometers. In one embodiment, at least about 90% of the particles have a mean of LD and SD that varies by about 10% or less from an average mean of LD and SD, where the average mean of LD and SD is between about 1 micrometer and about 30 micrometers. In one embodiment, at least about 90% of the particles have a mean of LD and SD that varies by about 10% or less from an average mean of LD and SD, where the average mean of LD and SD is between about 1 micrometer and about 25 micrometers. In one embodiment, at least about 90% of the particles have a mean of LD and SD that varies by about 10% or less from an average mean of LD and SD, where the average mean of LD and SD is between about 1 micrometer and about 20 micrometers. In one embodiment, at least about 90% of the particles have a mean of LD and SD that varies by about 10% or less from an average mean of LD and SD, where the average mean of LD and SD is between about 1 micrometer and about 10 micrometers. In one embodiment, at least about 90% of the particles have a mean of LD and SD that varies by about 10% or less from an average mean of LD and SD, where the average mean of LD and SD is between about 1 micrometer and about 5 micrometers.

In one embodiment, at least about 75% of the particles have a mean of LD and SD less than about 300 micrometers. In one embodiment, at least about 75% of the particles have a mean of LD and SD less than about 200 micrometers. In one embodiment, at least about 75% of the particles have a mean of LD and SD less than about 100 micrometers. In one embodiment, at least about 75% of the particles have a mean of LD and SD less than about 50 micrometers. In one embodiment, at least about 75% of the particles have a mean of LD and SD less than about 30 micrometers. In one embodiment, at least about 75% of the particles have a mean of LD and SD less than about 25 micrometers. In one embodiment, at least about 75% of the particles have a mean of LD and SD less than about 20 micrometers. In one embodiment, at least about 75% of the particles have a mean of LD and SD less than about 10 micrometers. In one embodiment, at least about 75% of the particles have a mean of LD and SD less than about 5 micrometers.

In one embodiment, at least about 90% of the particles have a mean of LD and SD less than about 300 micrometers. In one embodiment, at least about 90% of the particles have a mean of LD and SD less than about 200 micrometers. In one embodiment, at least about 90% of the particles have a mean of LD and SD less than about 100 micrometers. In one embodiment, at least about 90% of the particles have a mean of LD and SD less than about 50 micrometers. In one embodiment, at least about 90% of the particles have a mean of LD and SD less than about 30 micrometers. In one embodiment, at least about 90% of the particles have a mean of LD and SD less than about 25 micrometers. In one embodiment, at least about 90% of the particles have a mean of LD and SD less than about 20 micrometers. In one embodiment, at least about 90% of the particles have a mean of LD and SD less than about 10 micrometers. In one embodiment, at least about 90% of the particles have a mean of LD and SD less than about 5 micrometers.

A single material can be used as the porogen. Alternatively, two or more different porogen materials can be used.

### Implant Coating

The implants as disclosed herein can optionally be coated partially or entirely with a coating to control drug release. Implants can be dip-coated, spray-coated, pan-coated, or coated in a fluidized bed system. The coating can be applied by co-extrusion when implants are made by extrusion methods. However, if the coating is applied before removal of the porogen and loading of the pharmaceutical substance and optional excipient, then the coating should allow for removal of porogen from the implant and subsequent loading of the pharmaceutical substance and optional excipient. Thus the coating should either not cover the entire implant (for example, a co-extruded coating may only be applied as a stripe on the outside surface of the implant), or the coating should be permeable to a fluid which can remove the porogen, as well as the dissolved porogen in the removal fluid, so as to allow the porogen to be removed from the implant to create a porous structure, and should also be permeable to a solution of the pharmaceutical substance and optional excipient, so as to allow loading of the porous structure.

Rod-shaped implants can have a coating applied to their entire surface, followed by cutting small portions of the ends of each rod. This results in a partially coated rod, where the planar surfaces at each end of the rod have drug-containing matrix exposed, while the curved cylindrical sides are coated. If the coating is applied to the curved cylindrical sides by co-extrusion, cutting the extruded rod into pieces to form individual implant swill expose drug-containing matrix at each end of the rod.

The coating can be impermeable to the drug, in which case the coating should only partially cover the implant in order for drug to be released from the uncoated portion of the implant, or the coating should dissolve or degrade after a period of time to allow drug to be released from the newly-exposed drug-containing matrix. Alternatively, the coating can be permeable to the drug to a greater or lesser extent, allowing modulation of drug release.

### Manufacture of Loadable Porous Structures

In some embodiments, the loadable porous structures for preparation of the implants disclosed herein can be produced by blending particles of matrix material, such as a polymer, with particles of porogen of the desired size, and then extruding the blend. The blend mixture is heated to a temperature suitable for extrusion, such as the softening point of the matrix material (for example, the softening point of a polymer used as matrix material). At this point, optionally and if necessary, the softened mixture can be homogenized. The mixture is then extruded, e.g., via Microtruder screw extruder, Model No. RCP-025, Randcastle Extrusion Systems, Cedar Grove, N.J., or via other extrusion devices known in the industry. The diameter of extrusion, as well as temperature, pressure and other parameters can be controlled as appropriate for each matrix material and porogen.

The extruded mixture of matrix material and porogen, referred to as the extrudate, can be extruded horizontally and collected for further processing. The extrudate can be cut into desirable lengths, e.g., from about 1 to about 3 cm.

The extrudate is then treated with a porogen removal fluid or porogen removal fluids, which serve to remove the porogen from the extrudate, resulting in formation of a loadable porous structure. The material used for the extrudate should be insoluble in the fluid used to remove the porogen, so as not to dissolve the extrudate structure during removal of porogen. Examples of the treatment of the extrudate with porogen removal fluid include, but are not limited to, washing the extrudate with the fluid or fluids, or immersion of the extrudate in the fluid or fluids. While immersing the extrudate in the fluid or fluids, the fluid or fluids can be stirred, agitated, or sonicated to assist in removal of porogen. The fluids used to remove porogens are typically liquids or supercritical fluids. Examples of fluids which can be used for treating the extrudate to remove porogen include, but are not limited to, water, saline, aqueous buffers, alcohols such as ethanol or isopropanol, and supercritical carbon dioxide. Mixtures of water and alcohols can also be used, such as ethanol-water mixtures. Preferable fluids are 100% ethanol or water-ethanol mixtures.

Treatment of the extrudate with porogen removal fluid can be performed at atmospheric pressure (about 101,325 or about 100,000 Pascal), or under increased pressure, such as about 2 atmospheres (about 202,650 or about 200,000 Pa) of pressure to about 50 atmospheres (about 5,066,250 or about 5,000,000 Pa) of pressure, for example, at about 3 atm (about 303,997 or about 300,000 Pa), about 5 atm (about 506,625 or about 500,000 Pa), about 10 atm (about 1,013,250 or about 1,000,000 Pascal), about 20 atm (about 2,026,500 or about 2,000,000 Pa), or about 50 atm (about 5,066,250 or about 5,000,000 Pa), or in a range between any two of those values. Treatment of the extrudate with porogen removal fluid can be performed at room temperature or ambient temperature, or at elevated temperature, such as about 30° C. to about 200° C., about 50° C. to about 200° C., about 100° C. to about 200° C., about 30° C. to about 150° C., about 50° C. to about 150° C., or about 50° C. to about 100° C., provided that the temperature of the fluid used to remove the porogens should be below the melting temperature of the polymer used for the extrudate, so as not to adversely affect the structure of the extrudate.

In some embodiments, at least about 50%, at least about 60%, at least about 70%, at least about 80%, at least about 90%, at least about 95%, or at least about 99% of fluid-accessible porogen is removed from the extrudate by treating the extrudate with porogen removal fluid. In some embodiments, about 50% to about 99%, about 60% to about 99%, about 70% to about 99%, about 80% to about 99%, or about 90% to about 99% of fluid-accessible porogen is removed from the extrudate by treating the extrudate with porogen removal fluid. In some embodiments, substantially all of the fluid-accessible porogen is removed from the extrudate by treating the extrudate with porogen removal fluid. If the desired amount of porogen removal is not attained with a single treatment of the extrudate with porogen removal fluid, the treatment can be repeated as needed until the desired amount of porogen is removed.

Washing of the extrudate to remove the porogen may be followed by drying to remove any remaining fluid. Drying is typically done between about 30° C. and about 60° C. for about 6 to about 24 hours, such as at about 40° C. for about 12 hours.

The porosity and distribution of pore sizes in the porous structures formed can be measured by various techniques. Porous structures can be examined under optical microscopes or electron microscopes. Computed tomography (CT scans) can be used. Introduction of a liquid of known density into the structure and measurement of the weight gain of the porous structure allows calculation of the total volume of pores in the structure. Non-destructive techniques for measuring porosity include, but are not limited to, thermoporometry and cyroporometry (cryoporosimetry), such as differential scanning calorimetry thermoporometry, nuclear magnetic resonance cryoporometry (NMR cryoporosimetry), or neutron diffraction cryoporometry. Brunauer-Emmett-Teller (BET) analysis can be used to analyze available surface area of porous structures.

### Loading of Loadable Porous Structures to Form Loaded Porous Structures

Once the loadable porous structure is formed by removal of the porogens, the pharmaceutical substance(s) and optional excipient(s) can be loaded into the loadable porous structure to form the loaded porous structure, for use as the pharmaceutical substance-containing implant. The material to be loaded, whether one pharmaceutical substance or more than one pharmaceutical substance, and optionally in combination with one or more excipients, is referred to as the payload of the implant.

The payload can be dissolved in an appropriate solvent in which the payload can be solubilized, to form a payload solution. The loadable porous structure can then be loaded with the payload solution, followed by removing the solvent from the porous structure, leaving the payload in the pores of the structure and thus forming the loaded porous structure to be used as the implant. A preferred solvent is water. Organic solvents can be used, such as ethanol, ethyl acetate, dichloromethane, acetone, methanol, isopropanol, or any combination thereof. If an organic solvent is used, the solvent is preferably a Class 3 solvent as listed in the guidance from the United States Food and Drug Administration at URL www.fda.gov/downloads/drugs/guidances/ucm073395.pdf (which include ethanol, acetone, and ethyl acetate, among others); however, Class 2 solvents (which include dichloromethane and methanol, among others) can be used if necessary. Class 1 and Class 4 solvents should be used only when the payload cannot be dissolved in a suitable Class 3 or Class 2 solvent. Solvents which would damage or dissolve the matrix material should be avoided. Combinations of solvents, such as water/organic solvent such as water/ethanol, can also be used. Supercritical carbon dioxide can also be used as a solvent for the payload when applicable.

The solvent used to load the payload can then be removed by evaporation, lyophilization, or any other suitable method.

If insufficient payload has been loaded into the loadable porous structure by a single cycle of loading the loadable porous structure with payload solution and removing the solvent, then additional cycles of loading the loadable porous structure with payload solution and subsequently removing the solvent can be repeated until a predetermined amount of payload has been loaded into the structure. For example, if the predetermined amount of payload desired in the structure is 100 mg, and the amount of payload deposited in the structure during each cycle of loading payload solution and removing solvent is 25 mg, then four cycles of loading and removing should be performed.

Optionally, the loaded porous structure can be washed to remove pharmaceutical substance from the surface of the structure, to reduce burst release. Examples of solvents which can be used for washing the loaded porous structure include, but are not limited to, water, saline, aqueous buffers, and alcohols such as ethanol or isopropanol. Mixtures of water and alcohols can also be used, such as ethanol-water mixtures. Preferable solvents are 100% ethanol or water-ethanol mixtures. The loaded porous structure can then be dried to remove wash solvent, for example, at a temperature between about 30° C. and about 60° C. for about 6 to about 24 hours, such as at about 40° C. for about 12 hours.

Drying may be followed by packaging and sterilization to prepare the implants. Loaded porous structures for use as implants may be vacuum-packed in moisture barrier foil pouches, heat-sealed and/or vacuum-sealed, and then sterilized using gamma irradiation, such as about 20 to 30 kilograys, or about 25 kilograys, or about 2.5 to about 3.5 Megarad, or about 2.9 to about 3.1 Mrads, or about 3 Mrads.

### Insertion and Removal of Implants

Another aspect of this disclosure is a method for delivering a pharmaceutical substance or drug to a patient in need thereof, comprising the step of inserting an implant or implants as disclosed herein into the patient, wherein the pharmaceutical substance or drug is released from the implant or implants into the patient. In a preferred method of this disclosure, implants as disclosed herein are administered by subdermal implantation. In various embodiments, the implants are subdermally implanted at a site selected from a group consisting of the upper arm, scapular region, the back, the leg and the abdomen. Before implantation, the patient may be lightly anesthetized, e.g., with isoflurane or other anesthetic known in the art, and/or may have topical, transdermal, or subdermal anesthetic applied at the site of implantation. A small incision can be made through the skin and a trocar inserted subdermally, then loaded with one implant. The stylet can be inserted to hold the implant in place and the trocar carefully removed, leaving the implant in the subdermal space. Each site can be sutured closed and examined later. Complications such as skin irritation, inflammation, infection or other site-specific adverse effects can be monitored and treated, e.g., with antibiotics, as needed.

In various embodiments, implants as disclosed herein can be left in the body for up to about one year, about two years, or longer. The implants can be left in the body for up to about 3 months, up to about 6 months, up to about 9 months, up to about 12 months, up to about 15 months, up to about 18 months, up to about 21 months, or up to about 24 months or longer. The period of sustained release of drug into the body is thus from about 1 month to about 1 year, or longer, or from about 3 months to about 1 year or longer, e.g., at least about 3 months, at least about 6 months, at least about 9 months, at least about 12 months, at least about 15 months, at least about 18 months, at least about 21 months, or at least about 24 months or longer. In some embodiments the implants can be left in the body for more than 1 year. Implants may be removed from the body at the end of the treatment period, through an incision, e.g., a 3-mm incision, using forceps.

The implants as disclosed herein are configured such that, after implantation into a patient, the implants release drug for up to about 3 months, up to about 6 months, up to about 9 months, up to about 12 months, up to about 15 months, up to about 18 months, up to about 21 months, or up to about 24 months or longer. The implants are configured such that, after implantation into a patient, the period of sustained release of drug into the body is from about 1 month to about 1 year, or longer, or from about 3 months to about 1 year or longer, e.g., at least about 3 months, at least about 6 months, at least about 9 months, at least about 12 months, at least about 15 months, at least about 18 months, at least about 21 months, or at least about 24 months or longer.

A second implant may, for example, be used to deliver a pharmaceutical substance to counteract any adverse effects caused by a drug released from a first implant.

Multiple implants may be inserted into a single patient to regulate the delivery of a single drug, or to deliver several drugs.

The implants as disclosed herein can, after implantation into a patient, release drug at a steady-state level for up to about 3 months, up to about 6 months, up to about 9 months, up to about 12 months, up to about 15 months, up to about 18 months, up to about 21 months, or up to about 24 months or longer. The implants are configured such that, after implantation into a patient, the period of steady-state release of drug into the body is from about 1 month to about 1 year, or longer, or from about 3 months to about 1 year or longer, e.g., at least about 3 months, at least about 6 months, at least about 9 months, at least about 12 months, at least about 15 months, at least about 18 months, at least about 21 months, or at least about 24 months or longer. Steady-state may be attained after an initial period, such as after about one, two, three, four, five, six, or seven days after implantation.

The implants as disclosed herein can, after implantation into a patient, provide a constant plasma level of drug or approximately constant plasma level of drug for up to about 3 months, up to about 6 months, up to about 9 months, up to about 12 months, up to about 15 months, up to about 18 months, up to about 21 months, or up to about 24 months or longer. The implants are conigured such that, after implantation into a patient, the implants provide a constant plasma level of drug or approximately constant plasma level of drug from about 1 month to about 1 year, or longer, or from about 3 months to about 1 year or longer, e.g., at least about 3 months, at least about 6 months, at least about 9 months, at least about 12 months, at least about 15 months, at least about 18 months, at least about 21 months, or at least about 24 months or longer. A constant plasma level of drug or approximately constant plasma level of drug may be attained after an initial period, such as after about one, two, three, four, five, six, or seven days after implantation.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed

All publications, patents, and patent applications cited herein are hereby incorporated by reference in their entirety.

What is claimed is:

1. A method of making a loadable porous structure, comprising:
extruding a mixture of a biocompatible matrix material and a porogen to form a matrix material-porogen extrudate, and
removing the porogen from the extrudate to form the loadable porous structure.

2. The method of claim 1, wherein the matrix material is a polymer.

3. The method of claim 2, wherein the polymer is a non-biodegradable polymer.

4. The method of claim 1, wherein the matrix material comprises a material selected from the group consisting of acrylics, agarose, alginate, cellulose ethers, collagen, copolymers containing poly(ethylene glycol) and polybutylene terephthalate segments (PEG/PBT) (PolyActive™), copolymers of poly(lactic) and glycolic acid, copolymers thereof with poly(ethylene glycol), derivatives and mixtures thereof, dextran, dextrose, elastin, epoxides, ethylene vinyl acetate (EVA copolymer), fluoropolymers, gelatin, hydroxypropylmethylcellulose, maleic anhydride copolymers, methyl cellulose and ethyl cellulose, non-water soluble cellulose acetate, non-water soluble chitosan, non-water soluble hydroxyethyl cellulose, non-water soluble hydroxypropyl cellulose, peptides, PLLA-poly-glycolic acid (PGA) copolymer (also known as poly-L-lactic acid-co-glycolic acid, or PLGA), poly (L-lactic acid), poly(2-ethoxyethyl methacrylate), poly(2-hydroxyethyl methacrylate), poly(2-methoxyethyl acrylate), poly(2-methoxyethyl methacrylate), poly(acrylamide), poly(alginic acid), poly(amino acids), poly(anhydrides), poly(aspartic acid), poly(benzyl glutamate), poly(beta-hydroxybutyrate), poly(caprolactone), poly(D,L-lactic acid), poly(D,L-lactide)(PLA), poly(D,L-lactide-co-caprolactone)(PLA/PCL) and poly(glycolide-co-caprolactone) (PGA/PCL), poly(D,L-lactide-co-glycolide) (PLA/PGA), poly(etherurethane urea), poly(ethyl glutamate-co-glutamic acid), poly(ethylene carbonate), poly(ethylene glycol), poly(ethylene-co-vinyl alcohol), poly(glutamic acid), poly(glutamic acid-co-ethyl glutamate), poly(glycolic acid), poly(glycolide-co-trimethylene carbonate) (PGA/PTMC), poly(hydroxypropyl methacrylamide), poly(imino carbonates), poly(leucine), poly(leucine-co-hydroxyethyl glutamine), poly(L-lactide-co-D,L-lactide) (PLLA/PLA), poly(L-lactide-co-glycolide)(PLLA/PGA), poly(lysine), poly(ortho esters), poly(orthoesters), poly(oxaamides), poly(oxaesters), poly(phosphate ester), poly(phosphazene), poly(phospho esters), poly(phosphoesters), poly(propylene carbonate), poly(propylene glycol), poly(pyrrole), poly(tert-butyloxy-carbonylmethyl glutamate), poly(tetramethylene glycol), poly(trimethylene carbonate), poly(ureas), poly(urethanes), poly(urethane-ureas), poly(vinyl alcohol), poly(vinyl alcohol-co-vinyl acetate), high molecular weight poly(vinylpyrrolidone) (PVP), poly[(97.5% dimethyl-trimethylene carbonate)-co-(2.5% trimethylene carbonate)], polyacrylic acid, polyalkylene oxides, polyamides, polycaprolactone (PCL) poly-(hydroxybutyrate-co-hydroxyvalerate) copolymer (PHBV), polycaprolactone (PCL), polycaprolactone co-butylacrylate, polydepsipeptides, polydioxanone (PDS), polyesters, polyethylene glycol, polyethylene oxide (PEO), polyethylene terephthalate (PET), polyglycolic acid and copolymers and mixtures thereof, poly(L-lactide) (PLLA), polyglycolic acid[polyglycolide (PGA)], polyhydroxybutyrate (PHBT) and copolymers of polyhydroxybutyrate, polyiminocarbonates, polylactic acid, polymethacrylic acid, polyolefins, polyphosphazene polymers, polypropylene fumarate, polysaccharides, hyaluronic acid, polytetrafluoroethylene (PTFE Teflon®), polyurethanes, silicones, tyrosine-derived polyarylates, tyrosine-derived polycarbonates, tyrosine-derived polyiminocarbonates, tyrosine-derived polyphosphonates, urethanes, polyamide, aliphatic polycarbonates, polyalkylcyanoacrylate, polyalkylene oxalates, polyanhydride, polycarboxylic acid, polyester, poly(hydroxybutyrate), polyimide, poly(iminocarbonate), polycaprolactone (PCL), poly-D,L-lactic acid (DL-PLA), polydioxanone, poly(glycolic acid), poly-L-lactic acid (L-PLA), poly-L-lactic acid-co-glycolic acid (PLGA), polyorthoester, polyphosphazenes, and polyphosphoester, poly(trimethylene carbonate), cellulose ester, polybutylene terephthalate, polycarbonate, polyester, polyether ether ketone, polyethylene-co-tetrafluoroethylene, polymethylmethacrylate, polyolefin, polypropylene, polysulfones, polytetrafluoroethylene, polyurethane, polyvinylchloride, polyvinylidene fluoride, silicone, ABS resins, acrylic polymers and copolymers, acrylonitrile-styrene copolymers, alkyd resins, carboxymethyl cellulose, ethylene-vinyl acetate copolymers, cellophane, cellulose butyrate, cellulose acetate butyrate, cellulose acetate, cellulose ethers, cellulose nitrate, cellulose propionate, copolymers of vinyl monomers with each other and olefins, ethylene-methyl methacrylate copolymers, epoxy resins, ethylene vinyl alcohol copolymer, poly(glyceryl sebacate), poly(glycolic acid-co-trimethylene carbonate), poly(hydroxybutyrate-co-valerate), poly(hydroxyvalerate), poly(lactide-co-glycolide), poly(propylene fumarate), poly(trimethylene carbonate), polyacrylonitrile, polyamides, Nylon 66, polycaprolactam, polycarbonates, polycyanoacrylates, polydioxanone, polyesters, polyethers, polyimides, polyisobutylene and ethylene-alphaolefin copolymers, polyoxymethylenes, polyphosphoester urethane, polyvinyl ketones, polyvinyl aromatics, polystyrene, polyvinyl esters, polyvinyl acetate, polyvinyl ethers, s polyvinyl methyl ether, polyvinylidene halides, vinylidene fluoride based homo- or co-polymer under the trade name Solef™ or Kynar™, polyvinylidene fluoride (PVDF), poly(vinylidene-co-hexafluoropropylene) (PVDF-co-HFP), polyvinylidene chloride, rayon, rayon-triacetate, silicones, vinyl halide polymers and copolymers, polyvinyl chloride, copolymers of these polymers with poly(ethylene glycol) (PEG), copolymers of poly(lactic) and glycolic acid, poly(anhydrides), poly(D,L-lactic acid), poly(D,L-lactide), poly(D,L-lactide-co-glycolide), poly(ethylene carbonate), poly(glycolic acid), poly(glycolide), poly(L-lactic acid), poly(L-lactide), poly(L-lactide-co-glycolide), poly(ortho esters), poly(oxaamides), poly(oxaesters), poly(phosphazenes), poly(phospho esters), poly(phosphoesters), poly(propylene carbonate), poly(trimethylene carbonate), poly(tyrosine derived carbonates), poly(tyrosine derived iminocarbonates), poly(tyrosine derived arylates), copolymers of these polymers with poly(ethylene glycol) (PEG), poly(ethylene-co-vinyl acetate) (EVA), polyvinylalcohol, polyurethanes, polycarbonate-based polyurethanes, and any combination or mixture of any two or more of the foregoing.

5. The method of claim 1, wherein the matrix material is ethylene vinyl acetate (EVA).

6. The method of any one of claims 1-5, wherein the porogen comprises a material selected from the group consisting of an alkyl cellulose, a hydroxyalkyl cellulose, ethylcellulose, methylcellulose, hydroxymethylcellulose, a fatty acid, stearic acid, palmitic acid, myristic acid, linoleic acid, a biocompatible salt, sodium chloride, calcium chloride, sodium phosphate, a solid organic acid, citric acid, a soluble polymer, and low molecular weight polyvinylpyrrolidone (low MW PVP).

7. The method of any one of claims 1-5, wherein the porogen comprises ethylcellulose or methylcellulose.

8. The method of any one of claims 1-7, wherein removing the porogen from the extrudate comprises treating the extrudate with a fluid that removes the porogen.

9. The method of claim 8, wherein the treating of the extrudate with the fluid comprises washing the extrudate with the fluid or immersing the extrudate in the fluid.

10. The method of claim 8 or claim 9, wherein the fluid comprises water, saline, an aqueous buffer, an alcohol, ethanol, isopropanol, or supercritical carbon dioxide.

11. The method of any one of claims 1-10, wherein at least about 50% of the fluid-accessible porogen is removed from the extrudate.

12. The method of any one of claims 1-11, wherein the porogen is not a pharmaceutically active substance or drug.

13. A loadable porous structure made by any one of the methods of claims 1-12.

14. A method of making a loaded porous structure, comprising:
forming a loadable porous structure by the method of any one of claims 1-12;
loading a payload solution into pores of the loadable porous structure, where the payload solution comprises a solvent, a pharmaceutical substance, and optionally an excipient; and
removing the solvent from the loadable porous structure to form the loaded porous structure.

15. The method of claim 14, further comprising repeating the steps of loading and removing until the loaded porous structure contains a predetermined amount of pharmaceutical substance and optional excipient.

16. The method of claim 14 or claim 15, wherein the pharmaceutical substance comprises a substance selected from the group consisting of a protein and a nucleic acid.

17. The method of any one of claims 14-16, wherein the optional excipient comprises a sugar alcohol, mannitol, glycerol, erythritol, threitol, arabitol, ribitol, xylitol, fucitol, galactitol, iditol, inositol, sorbitol, volemitol, isomalt, lactitol, maltitol, a biodegradable polymer, or poly (lactic-co-glycolic acid) (PLGA).

18. A loaded porous structure, made by any one of the methods of claims 14-17.

19. A loadable porous structure, said structure prepared by a method comprising:
extruding a mixture of a biocompatible matrix material and a porogen to form a matrix material-porogen extrudate; and
removing the porogen from the extrudate to form the loadable porous structure.

20. A loaded porous structure, comprising:
a loadable porous structure of claim 19, and a pharmaceutical substance loaded into the pores of the loadable porous structure.

21. The loaded porous structure of claim 20, wherein the pharmaceutical substance is combined with an excipient prior to loading into the pores of the loadable porous structure.

22. The loadable porous structure of claim 19, wherein the matrix material is a polymer.

23. The loaded porous structure of claim 20 or claim 21, wherein the matrix material is a polymer.

24. The loadable porous structure of claim 22 or the loaded porous structure of claim 23, wherein the polymer is a non-biodegradable polymer.

25. The loadable porous structure of claim 22 or the loaded porous structure of claim 23, wherein the matrix material comprises a material selected from the group consisting of acrylics, agarose, alginate, cellulose ethers, collagen, copolymers containing poly(ethylene glycol) and polybutylene terephthalate segments (PEG/PBT) (PolyActive™), copolymers of poly(lactic) and glycolic acid, copolymers thereof with poly(ethylene glycol), derivatives and mixtures thereof, dextran, dextrose, elastin, epoxides, ethylene vinyl acetate (EVA copolymer), fluoropolymers, gelatin, hydroxypropylmethylcellulose, maleic anhydride copolymers, methyl cellulose and ethyl cellulose, non-water soluble cellulose acetate, non-water soluble chitosan, non-water soluble hydroxyethyl cellulose, non-water soluble hydroxypropyl cellulose, peptides, PLLA-poly-glycolic acid (PGA) copolymer (also known as poly-L-lactic acid-co-glycolic acid, or PLGA), poly (L-lactic acid), poly(2-ethoxyethyl methacrylate), poly(2-hydroxyethyl methacrylate), poly(2-methoxyethyl acrylate), poly(2-methoxyethyl methacrylate), poly(acrylamide), poly(alginic acid), poly(amino acids), poly(anhydrides), poly(aspartic acid), poly(benzyl glutamate), poly(beta-hydroxybutyrate), poly(caprolactone), poly(D,L-lactic acid), poly(D,L-lactide)(PLA), poly(D,L-lactide-co-caprolactone)(PLA/PCL) and poly(glycolide-co-caprolactone) (PGA/PCL), poly(D,L-lactide-co-glycolide) (PLA/PGA), poly(etherurethane urea), poly(ethyl glutamate-co-glutamic acid), poly(ethylene carbonate), poly(ethylene glycol), poly(ethylene-co-vinyl alcohol), poly(glutamic acid), poly(glutamic acid-co-ethyl glutamate), poly(glycolic acid), poly(glycolide-co-trimethylene carbonate) (PGA/PTMC), poly(hydroxypropyl methacrylamide), poly(imino carbonates), poly(leucine), poly(leucine-co-hydroxyethyl glutamine), poly(L-lactide-co-D,L-lactide) (PLLA/PLA), poly(L-lactide-co-glycolide)(PLLA/PGA), poly(lysine), poly(ortho esters), poly(orthoesters), poly(oxaamides), poly(oxaesters), poly(phosphate ester), poly(phosphazene), poly(phospho esters), poly(phosphoesters), poly(propylene carbonate), poly(propylene glycol), poly(pyrrole), poly(tert-butyloxy-carbonylmethyl glutamate), poly(tetramethylene glycol), poly(trimethylene carbonate), poly(ureas), poly(urethanes), poly(urethane-ureas), poly(vinyl alcohol), poly(vinyl alcohol-co-vinyl acetate), high molecular weight poly(vinylpyrrolidone) (PVP), poly[(97.5% dimethyl-trimethylene carbonate)-co-(2.5% trimethylene carbonate)], polyacrylic acid, polyalkylene oxides, polyamides, polycaprolactone (PCL) poly-(hydroxybutyrate-co-hydroxyvalerate) copolymer (PHBV), polycaprolactone (PCL), polycaprolactone co-butylacrylate, polydepsipeptides, polydioxanone (PDS), polyesters, polyethylene glycol, polyethylene oxide (PEO), polyethylene terephthalate (PET), polyglycolic acid and copolymers and mixtures thereof, poly(L-lactide) (PLLA), polyglycolic acid[polyglycolide (PGA)], polyhydroxybutyrate (PHBT) and copolymers of polyhydroxybutyrate, polyiminocarbonates, polylactic acid, polymethacrylic acid, polyolefins, polyphosphazene polymers, polypropylene fumarate, polysaccharides, hyaluronic acid, polytetrafluoroethylene (PTFE Teflon®), polyurethanes, silicones, tyrosine-derived polyarylates, tyrosine-derived polycarbonates, tyrosine-derived polyiminocarbonates, tyrosine-derived polyphosphonates, urethanes, polyamide, aliphatic polycarbonates, polyalkylcyanoacrylate, polyalkylene oxalates, polyanhydride, polycarboxylic acid, polyester, poly(hydroxybutyrate), polyimide, poly(iminocarbonate), polycaprolactone (PCL), poly-D,L-lactic acid (DL-PLA), polydioxanone, poly(glycolic acid), poly-L-lactic acid (L-PLA), poly-L-lactic acid-co-glycolic acid (PLGA), polyorthoester, polyphosphazenes, and polyphosphoester, poly(trimethylene carbonate), cellulose ester, polybutylene terephthalate, polycarbonate, polyester, polyether ether ketone, polyethylene-co-tetrafluoroethylene, polymethylmethacrylate, polyolefin, polypropylene, polysulfones, polytetrafluoroethylene, polyurethane, polyvinylchloride, polyvinylidene fluoride, silicone, ABS resins, acrylic polymers and copolymers, acrylonitrile-styrene copolymers, alkyd resins, carboxymethyl cellulose, ethylene-vinyl acetate copolymers, cellophane, cellulose butyrate, cellulose acetate butyrate, cellulose acetate, cellulose ethers, cellulose nitrate, cellulose propionate, copolymers of vinyl monomers with each other and olefins, ethylene-methyl methacrylate copolymers, epoxy resins, ethylene vinyl alcohol copolymer, poly(glyceryl sebacate), poly(glycolic acid-co-trimethylene carbonate), poly(hydroxybutyrate-co-valerate), poly(hydroxyvalerate), poly(lactide-co-glycolide), poly(propylene fumarate), poly(trimethylene carbonate), polyacrylonitrile, polyamides, Nylon 66, polycaprolactam, polycarbonates, polycyanoacrylates, polydioxanone, polyesters, polyethers, polyimides, polyisobutylene and ethylene-alphaolefin copolymers, polyoxymethylenes, polyphosphoester urethane, polyvinyl ketones, polyvinyl aromatics, polystyrene, polyvinyl esters, polyvinyl acetate, polyvinyl ethers, s polyvinyl methyl ether, polyvinylidene halides, vinylidene fluoride based homo- or co-polymer under the trade name Solef™ or Kynar™, polyvinylidene fluoride (PVDF), poly(vinylidene-co-hexafluoropropylene) (PVDF-co-HFP), polyvinylidene chloride, rayon, rayon-triacetate, silicones, vinyl halide polymers and copolymers, polyvinyl chloride, copolymers of these polymers with poly(ethylene glycol) (PEG), copolymers of poly(lactic) and glycolic acid, poly(anhydrides), poly(D,L-lactic acid), poly(D,L-lactide), poly(D,L-lactide-co-glycolide), poly(ethylene carbonate), poly(glycolic acid), poly(glycolide), poly(L-lactic acid), poly(L-lactide), poly(L-lactide-co-glycolide), poly(ortho esters), poly(oxaamides), poly(oxaesters), poly(phosphazenes), poly(phospho esters), poly(phosphoesters), poly(propylene carbonate), poly(trimethylene carbonate), poly(tyrosine derived carbonates), poly(tyrosine derived iminocarbonates), poly(tyrosine derived arylates), copolymers of these polymers with poly(ethylene glycol) (PEG), poly(ethylene-co-vinyl acetate) (EVA), polyvinylalcohol, polyurethanes, polycarbonate-based polyurethanes, and any combination or mixture of any two or more of the foregoing.

26. The loadable porous structure of claim 22 or the loaded porous structure of claim 23, wherein the matrix material is ethylene vinyl acetate (EVA).

27. The loadable porous structure of any one of claim 19, 22, or 24-26, wherein the porogen comprises a material selected from the group consisting of an alkyl cellulose, a hydroxyalkyl cellulose, ethylcellulose, methylcellulose, hydroxymethylcellulose, a fatty acid, stearic acid, palmitic acid, myristic acid, linoleic acid, a biocompatible salt, sodium chloride, calcium chloride, sodium phosphate, a solid organic acid, citric acid, a soluble polymer, and low molecular weight polyvinylpyrrolidone (low MW PVP).

28. The loadable porous structure of any one of claim 19, 22, or 24-26, wherein the porogen is not a pharmaceutically active substance or drug.
